(19) United States
(12) Patent Application Publication
(10) Pub. No.: US 2007/0080305 A1
(43) Pub. Date: Apr. 12, 2007

(54) Device and process for luminescence imaging

(75) Inventors: Serge Maitrejean, Paris (FR); Pascal Asselin, Grandfresnoy (FR); Emilie Roncali, Paris (FR); Bertrand Tavitian, Paris (FR)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BIOSPACE MESURES, Paris (FR)

(21) Appl. No.: 11/284,272

(22) Filed: Nov. 21, 2005

(30) Foreign Application Priority Data
Oct. 10, 2005 (FR) 05 10332

(51) Int. Cl. G01J 1/58; A61B 6/00; F21V 9/16; G01T 1/10; G21H 3/02; G21K 5/00

(52) U.S. Cl. 250/458.1; 600/476

(57) ABSTRACT

The luminescence imaging apparatus comprises:
a stage receiving a sample (2) emitting luminescence information about the sample;
a light source (8) illuminating the stage; and an electronic control unit defining time frames; a combined light signal corresponding to a combination of luminescence information and of the reflection on the sample of the illumination.

During each time frame, the detection apparatus (9) acquires both first data relating to the luminescence information, and also second data relating to the second light signal.

### TECHNICAL FIELD

The present invention relates to methods and apparatus for luminescence imaging.

### BACKGROUND OF THE INVENTION

More particularly, the invention relates mainly to luminescence imaging apparatus comprising:
a light-tight enclosure containing a stage adapted to receive a sample that is to be imaged and that emits a first light signal carrying luminescence information about the sample;
a light source adapted to generate incident illumination towards the stage, the interaction between said incident illumination and the sample forming a second light signal;
detection apparatus adapted firstly to detect light signals presenting a luminescence spectrum and to store a first image on the basis of the light signals presenting a luminescence spectrum, and secondly to detect light signals corresponding to the reflection on the sample of the incident illumination coming from said light source and to store a second image on the basis of the light signals corresponding to the reflection on the sample of the incident illumination coming from said light source; and
an electronic control unit adapted to define a plurality of time frames, each time frame lasting for a length of time corresponding to acquiring and storing the second image;
said electronic control unit also being adapted to cause the light source to generate incident illumination during each time frame; and
a combined light signal corresponding to a combination of the first and second light signals reaching the detection apparatus during each time frame.

Document WO 01/37,195 describes an example of such apparatus. That apparatus has a “live” mode for taking a plurality of photographic representations of the sample. Then, when the sample emits light due to a chemical reaction taking place inside the sample (the luminescence phenomenon), that apparatus can take luminescence images of the sample, thereby detecting the quantity of light emitted by the sample due to the chemical reactions in question.

However, that apparatus does not make it possible to monitor rapid variation over time of the information relating to the luminescence. If the sample moves while the measurement is taking place (in particular if it is necessary for the sample to move while the measurement is taking place because the measurement corresponds to muscular activity that cannot be recorded for a anesthetized sample), such an installation is not suitable.

An object of the present invention is to provide apparatus making it possible to mitigate those drawbacks.

### SUMMARY OF THE INVENTION

To this end, according to the invention, apparatus of the type in question is characterized in that it further comprises separator means adapted so that, during each time frame, the detection apparatus acquires both first data relating to the luminescence information, and also second data relating to the second light signal.

By means of these provisions, information corresponding to a cinematographic representation of the sample and information relating to the luminescence of the sample are acquired simultaneously at the scale of the video frame time.

In certain embodiments of the invention, it is optionally possible to use one or more of the following provisions:
the detection apparatus has a plurality of pixels, each of which is adapted to detect light signals coming from a respective given region of the enclosure;
the detection apparatus is adapted to store the first image on the basis of a first sampling of time frames and to store the second image on the basis of a second sampling of time frames, the first sampling having a frequency that is different from the frequency of the second sampling;
the frequency of the first sampling is lower than the frequency of the second sampling;
the second light signal comprises a light signal relating to the reflection on the sample of the incident illumination coming from said light source, and a light signal of autofluorescence of the sample subjected to said incident illumination, and the imaging apparatus is adapted to separate the light signals presenting a luminescence spectrum from the light signal of autofluorescence and from the light signal relating to the reflection;
the detection apparatus comprises:
a first detector adapted to detect light signals carrying luminescence information; and
a second detector adapted to detect light signals corresponding to the reflection on the sample of the incident light coming from said light source;
the separator means comprise a filter disposed at the inlet of the first detector, said filter being adapted to ensure that the light signals corresponding to the reflection on the sample of the incident light coming from said light source are not acquired by the first detector;
the separator means further comprise a separator plate adapted to transmit the first light signal to the first detector, and to transmit the second light signal to the second detector;
the first and second detectors are offset angularly relative to each other, and each of said detectors receives the combined light signal directly, the imaging apparatus further comprising a reconstruction unit adapted to associate the first data and the second data with a reference frame associated with the enclosure;
the light source emits continuously, and the combined light signal is a spectral combination of the first and second light signals;
the first light signal presents a spectrum distributed between a shortest wavelength and a longest wavelength, and the light source emits an incident illumination distributed substantially beyond said longest wavelength;
the separator means comprise a sequencer adapted so that the control unit causes the light source to generate said incident illumination in pulsed manner, each time frame presenting an ON time, during which the light source emits, and an OFF time, during which the light source does not emit,
the combined light signal being a temporal combination of the first and second light signals,
the sequencer being adapted to cause the first detector to be in a detection state during the OFF time, and to be in a non-detection state during the ON time; and
a processor unit adapted to transpose the luminescence information to a reference frame associated with the sample.

In another aspect, the invention provides a method of performing luminescence imaging, said method comprising the following steps:

with a light-tight enclosure containing a stage receiving a sample that is to be imaged and that emits a first light signal carrying luminescence information about the sample,

(a) having an electronic control unit define a plurality of time frames, and having said control unit cause the light source to generate incident illumination towards the stage during each time frame, the interaction between said incident illumination and the sample forming a second light signal;

a combined light signal corresponding to a combination of the first and second light signals reaching detection apparatus during each time frame;

(b) separating the combined light signal so that, during each time frame, the detection apparatus, which is adapted firstly to detect light signals presenting a luminescence spectrum and to store a first image on the basis of the light signals presenting a luminescence spectrum, and secondly to detect light signals corresponding to the reflection on the sample of the incident illumination coming from said light source and to store a second image on the basis of the light signals corresponding to the reflection on the sample of the incident illumination coming from said light source, acquires both first data relating to the luminescence information, and also second data relating to the second light signal, each time frame lasting for a length of time corresponding to acquiring and storing a second image.

In certain implementations of the invention, it is optionally possible to use one or more of the following provisions:
during step (a), each time frame is subdivided into an ON time during which the light source emits, and an OFF time during which the light source does not emit;

the combined light signal being a temporal combination of the first and second light signals;

and, during step (b), a first detector adapted to detect light signals presenting a luminescence spectrum is caused to be in a detection state during the OFF time, and to be in a non-detection state during the ON time, and a second detector adapted to detect light signals corresponding to the reflection on the sample of the incident illumination is caused to be in a detection state at least during the ON time;
during step (b), a first detector detects the first light signal carrying the luminescence information, and a second detector detects the light signal corresponding to the reflection on the sample of the incident light coming from said light source, while ensuring that the light signals corresponding to the reflection on the sample of the incident light coming from said light source do not reach the first detector;
the detection apparatus has a plurality of pixels, and, during each time frame, the first data and the second data is associated with co-ordinates of a region of the enclosure;
for each time frame, the first data is transposed to a reference frame associated with the sample;
prior to step (a), a chemical reaction is triggered inside the sample, said chemical reaction generating the first light signal, and, after step (b) information relating to the chemical reaction is extracted from the first data and the second data;
prior to step (a), the method further consists in:
illuminating at least one molecule adapted to emit a phosphorescence signal due to it being illuminated, and
inserting the illuminated molecule into the sample,
the first light signal corresponding to phosphorescence light emitted by the molecule from inside the sample.

In another aspect, the invention provides a method of performing luminescence imaging, said method comprising the following steps:

(c) placing a sample to be imaged on a stage in a light-tight enclosure, said sample having an outside surface defining an inside, the inside of said sample emitting a light signal into the enclosure through the outside surface, the sample also emitting a second signal of a type different from the type of the light signal;

(d) for a period of observation throughout which the sample is contained in the enclosure, detecting said light signal and said second signal;

said method further comprising the following steps:

(e) on the basis of the detection of the light signal, and for at least first and second successive time frames of the period of observation, forming a luminescence image of the sample representing the light signal emitted from inside the sample; and

(f) on the basis of the detection of the second signal, and for at least said first and second time frames, forming a cinematographic image of the sample that represents the position of the sample in the enclosure.

In certain implementations of the invention, it is optionally possible to use one or more of the following provisions:
for each time frame, a luminescence image of the sample is formed directly by detecting the light signal; and
for each time frame, a cinematographic image of the sample is formed directly by detecting the second signal;
for each time frame, a cinematographic image of the sample is formed directly by detecting the second signal,
during a sub-period of detection, included in the detection period, and including at least the first and second time frames, an accumulated light signal is detected, and
said luminescence image is formed for each time frame by processing said light signal on the basis of said corresponding cinematographic images;
for at least one time frame, and preferably for each time frame, the method further consists in:

(g) displaying on a screen a superimposed image corresponding to the superposition of the cinematographic image and of the luminescence image for said time frame;
for at least one time frame, and preferably for each time frame, the method further consists in:

(h) identifying a region of the luminescence image, and

(i) by means of at least one cinematographic image, associating said region with a zone of the sample;
for said at least one time frame, and preferably for each time frame, the method further consists in (j) obtaining a parameter relating to a chemical entity located in said zone on the basis of the luminescence image;
during step (d), the light signal is detected at a first angle of incidence, and the second signal is detected at a second angle of incidence that is distinct from the first angle of incidence, and, for each time frame during steps (e) and (f), the luminescence image and the second image are formed in the same reference frame;
each image comprises a plurality of pixels (x, y) each of which corresponds to a zone (Du, Dv, Dw) of the enclosure;
the luminescence signal presents a spectrum, and the second signal is a light signal presenting a spectrum remote from the spectrum of the luminescence signal;
each time frame presents an ON time during which the sample is illuminated and during which the second signal is detected, and an OFF time during which the sample is not illuminated and during which the first signal is detected, and the second signal is a light signal corresponding to the reflection on the sample of the illumination;
the second signal is a thermal signal; and
each time frame lasts for a length of time corresponding to the detection time of the second signal.

### BRIEF DESCRIPTION OF THE FIGURES

Other characteristics and advantages of the invention appear from the following description of three of embodiments thereof given by way of non-limiting example, and with reference to the accompanying drawings.

In the drawings:

FIG. 1 is a diagrammatic perspective view of imaging apparatus;

FIG. 2 is a diagrammatic plane view of the inside of the enclosure of a first embodiment of the apparatus of FIG. 1;

FIG. 3 is a block diagram of an example of processing the data;

FIG. 4 is a diagram showing an example of the processing performed by the processor unit of FIG. 3;

FIGS. 5a, 5b, and 5c are graphs showing the states respectively of the light source, of the second detector and of the first detector, in a variant embodiment of the invention;

FIG. 6 is a view corresponding to FIG. 2 for a second embodiment of the invention; and

FIG. 7 is a view corresponding to FIG. 2 for a third embodiment of the invention.

### DETAILED DESCRIPTION OF THE INVENTION

In the various figures, like references designate elements that are identical or similar.

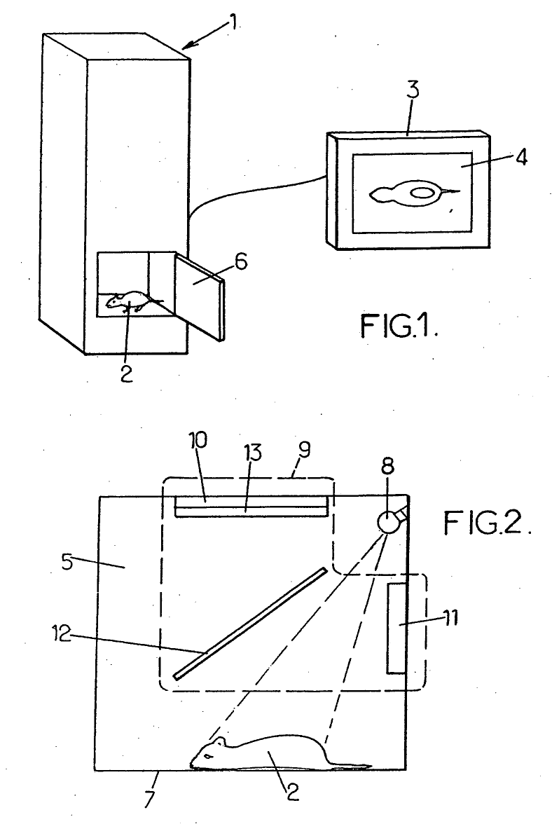

FIG. 1 diagrammatically shows imaging apparatus 1 designed to take an image of a sample 2, and a viewing screen 3 comprising a display 4 showing an image of the sample 2.

The imaging apparatus described herein is luminescence imaging apparatus, e.g. bioluminescence imaging apparatus, i.e. designed to take an image of a sample 2, such as, in particular, a small laboratory animal, e.g. a mammal, emitting light from inside its body.

For example, said light is generated due to a chemical reaction inside the body of the small animal. In order to obtain the chemical reaction, it is possible, for example, to take a small laboratory animal that has been genetically modified to include a gene encoding for a protein that presents the particularity of emitting light when it reacts chemically with a given complementary chemical entity, such as a molecule, an atom, or an ion.

Before placing the laboratory animal 2 in the imaging apparatus 1, said complementary molecule is given to it, e.g. by inoculation, and, optionally, time is left to enable the molecule to reach the possible site of reaction with the protein. The quantity of light given off locally by the chemical reaction is representative of the quantity of protein produced, and thus makes it possible to measure locally the level of expression of the gene.

In particular, if it is desired to check whether the gene in question is expressed particularly in response to a given event, it is possible to implement the measurement explained below firstly for a small laboratory animal 2 for which the event has been triggered, and secondly for a small laboratory animal 2 for which the event has not been triggered, in order to compare the signals emitted by the two animals.

Alternatively, the experiment in question can, for example, consist in measuring the muscular activity generated by an event in a laboratory animal, by detecting the quantity of light emitted by the coelenterazine-aequorin substrate-photoprotein pair which reacts with a given complementary chemical entity. For example, the entity in question is calcium arriving in the proximity of the photoprotein at the axons.

Since such events have a very fast time signature, it is useful to obtain information relating to the reaction rate rapidly.

The apparatus described herein can also be used to implement a method of performing imaging by delayed luminescence or phosphorescence. During such a method, a molecule adapted to emit light by phosphorescence for a time that is sufficiently long, of the order of a few minutes, is illuminated ex-vivo in order to trigger said phosphorescence. The molecule is then introduced into a small laboratory animal and can be used as a light tracer. The concentration of the molecule in a location of the organism, e.g. because a certain reaction takes place at that location, and because the molecule in question participates in said reaction, is detectable by the apparatus described below and makes it possible to characterize the reaction in question quantitatively or qualitatively.

As shown in FIGS. 1 and 2, the small laboratory animal 2 is placed in an enclosure 5 that is made light-tight, e.g. by closing a door 6 or the like. As shown in FIG. 2, the enclosure has a stage 7 which, for example, is formed by the floor of the enclosure, and on which the small laboratory animal 2 is disposed, and a light source 8 generating incident illumination towards the stage 7 (e.g. conveyed by an optical fiber).

Due to the above-described reaction, the small laboratory animal 2 naturally emits a first light signal that carries information relating to the luminescence of the small animal. In addition, due to the illumination generated by the light source 8, a second light signal, corresponding substantially to the incident illumination 8 being reflected by the small laboratory animal 2 is also emitted in the enclosure 5. Said second light signal can also include a portion corresponding to the autofluorescence of the sample 2 due to the illumination by the light source 8.

Said first and second light signals combine to form a combined light signal arriving at detection apparatus 9 shown outlined in dashed lines in FIG. 2.

In the first embodiment shown with reference to FIG. 2, the detection apparatus comprises a first detector 10 suitable for detecting light signals coming from the sample 2 that present a luminescence spectrum. Such a first detector 10 is, for example, a cooled charge-coupled device (CCD) camera presenting a matrix of pixels disposed in rows and in columns, an intensified CCD (ICCD), an electron multiplying CCD (EMCCD, i.e. a CCD with internal multiplication) or the like. The detection apparatus 9 further comprises a second detector 11 which, for example, is a conventional or an intensified CCD camera, presenting a large number of pixels disposed in rows and in columns. In the example shown in FIG. 2, each of the first and second detectors 10, 11 is disposed on a distinct face of the enclosure 5.

In the example shown, the light source 8 emits incident illumination continuously towards the stage so that the combined light signal corresponds to a spectral combination of the first light signal (carrying the luminescence information) and of the second light signal. The combined light signal is separated by a separator plate 12, which separates the signals on the basis of their wavelengths. For example, such a separator plate is a dichroic mirror or a mirror of the “hot mirror” type that separates visible from infrared. The light signal carrying the luminescence information is transmitted substantially in full towards the first detector 10, whereas the second light signal is transmitted substantially in full to the second detector 11.

In order to be sure that only the signal carrying the luminescence information reaches the first detector 10, it is also possible to dispose a filter 13 at the inlet of the first detector 10, which filter is adapted to prevent the wavelengths that do not correspond to that signal from reaching the first detector 10.

In practice, in order to be certain that the signal reaching the first detector 10 corresponds only to the luminescence from the inside of the sample 2, provision is made for the autofluorescence signal emitted by the sample 2 under the effect of the light source 8 to present a wavelength that is different from the wavelength of the signal in question. To this end, it is possible to choose to work with a light source 8 that emits incident illumination presenting an adapted spectrum, distributed beyond the range of wavelengths emitted by luminescence. For example, it is possible to use infrared illumination centered on a wavelength substantially equal to 800 nanometers (nm) when the luminescence spectrum presents a longest wavelength of 700 nm or shorter.

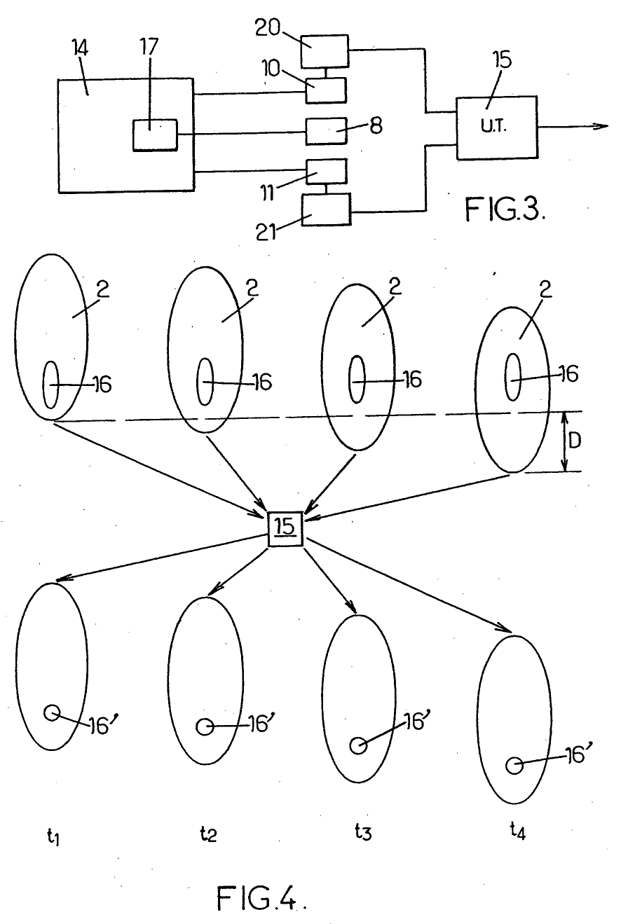

As shown in FIG. 3, an electronic control unit 14 is disposed that defines a plurality of time frames, each of which lasts a few milliseconds, corresponding substantially to the time necessary to acquire and to store a cinematographic representation of the stage 7 by means of the second detector 11. This cinematographic representation comprises a plurality of data pairs comprising co-ordinates and a light property (brightness, etc.). It is possible to set said time frames to have a time determined by the user, if said user desires a given acquisition rate, e.g. such as 24 images per second, or some other rate. At the start of each time frame, the preceding signal generated in the second detector 11 is read and stored in a second memory 21, as are the co-ordinates relating to each pixel, and another acquisition starts at the second detector 11.

In similar manner, at the start of each time frame, the signal generated by the first detector 10 is stored in a first memory 20 as are the co-ordinates relating to each pixel. A processor unit 15 is adapted to read the data stored in the first and second memories 20, 21, so as store it and/or so as to display the corresponding images on the display 4.

However, it can happen that it is preferable not to read the data measured at the first detector 10 for each time frame, but rather once every n time frames, where n is greater than 1, in order to allow signal to accumulate at the first detector 10 so that this signal is sufficiently strong to be able to be detected. For example, reading of the first detector 10 is triggered only about every 0.3 seconds, which remains a time that is relatively short relative to the speed of the observed phenomena. In which case, it is, for example, possible to make provision for the processor unit 15 to be adapted to re-compute, for each photographic representation acquired by the second detector 11, a value representative of the luminescence information for each of said representations, e.g. in the manner shown diagrammatically in FIG. 4.

FIG. 4 shows, at the top, four images of the sample 2 that are acquired successively by the second detector 11 at successive times T1, T2, T3, and T4. As is shown roughly in FIG. 4, the sample 2 might move forwards from instant T1 to instant T4, by a given distance D, which is intentionally exaggerated in FIG. 4 for explanatory purposes.

An image represented by reference 16, carrying luminescence information and obtained by the first detector 10, is superposed on each image of the sample 2 obtained by the second detector 11. Because, from time T1 to T4, a single acquisition has been performed at the first detector 10, the same image 16 is obtained for all four of those instants of the top of FIG. 4, that image appearing blurred because it corresponds to an emission zone of the sample 2 that has moved between the instants T1 and T4, due to the sample itself moving.

Once the four images coming from the second detector 11 for the four instants T1, T2, T3, and T4, and the image coming from the first detector 10 for the instant lasting from T1 to T4 have all been recorded, the processor unit 15 can, on the basis of the four photographic representations delivered by the second detector 11, compute the location, represented at 16′ on the photographic representations of FIG. 4, of the zone of the sample 2 that is emitting the luminescence information. For example, the displacement field to which the sample 2 has been subjected, is extracted from the four photographic representations delivered by the second detector 11, e.g. by using shape recognition on the photographic representations. Then, processing is applied to the image obtained by the first detector 10, said processing making it possible, from the single image, to obtain four identical probable images corresponding to respective ones of the instants T1, T2, T3, and T4. Then, the four identical probable luminescence images are superposed on the four photographic representations from the second detector 11 in order to deliver the succession of images shown at the bottom of FIG. 4.

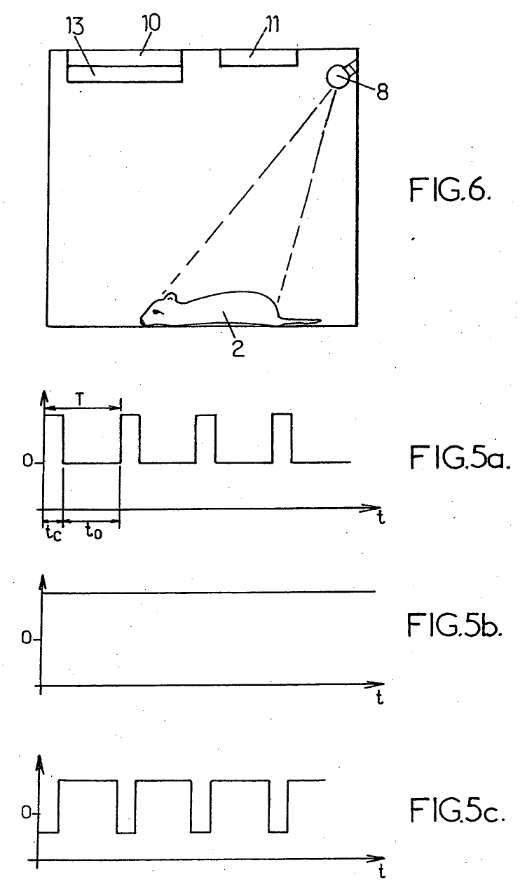

The embodiment shown with reference to FIG. 2 puts some constraint on the light source 8 because said light source must illuminate the sample 2 in a range of wavelengths such that the autofluorescence of the sample 2 due to said illumination presents a spectrum remote from the luminescence emission spectrum of the sample 2. In a variant embodiment explained below with reference to FIGS. 5a and 5c, it is also possible to use illumination that is pulsed at about video frequency. Such illumination is, for example, delivered from a laser diode, or the like. In this variant embodiment, the electronic control unit includes a sequencer 17 which causes the light source 8 to generate the incident illumination for an ON time t_{c} of the time frame T. Said incident illumination is, for example, synchronized with acquisition by the detector of the luminescence signal. It should be noted that, in the preceding embodiment, the electronic control unit causes the light source to generate the incident illumination continuously, and therefore, during all of the time frames. In the present variant, during the ON time t_{c}, e.g. situated at the start of the frame T, incident illumination is emitted towards the stage, so that a light signal comprising mainly a reflection of the incident illumination by the sample 2 reaches the detection apparatus 9.

As shown in FIG. 5c, the first detector 10 is then blinded, so that said first detector cannot detect any signal. In order to shield the first detector 10, it is possible to use a mechanical shutter situated at the inlet of the first detector 10, or electrical shielding is obtained, e.g. by reversing the voltage across the terminals of the first detector. Then, at the end of time t_{c}, the electronic control unit causes the incident illumination to be switched OFF, so that a few instants after t_{c}, only the luminescence coming from the sample 2 is detectable in the enclosure 5. During this OFF time t₀, the first detector 10 is once again in the detection state, and it detects the light signal carrying the luminescence information coming from the sample 2. In this variant embodiment, during one time frame, the combined light signal thus corresponds substantially to a temporal combination of the first and second signals, the (luminescent) first light signal being in the majority during the OFF time, and the second light signal, corresponding to the photographic representation of the sample, being in the majority during the ON time t_{c}. It should be noted that, because of the respective levels of the signals, the fact that, throughout the entire time frame, the sample 2 also emits a signal carrying the luminescence information, has no influence over the signal detected by the second detector 11. Indeed, the second detector can remain in acquisition mode during the OFF time t_{o,} as shown in FIG. 5b, without any significant influence on the measurement performed by said detector.

For the light source 8, in the above-described variant embodiment, it is also possible to use a light source 2 of spectrum targeted on 800 nanometers, as in the first embodiment.

However, it is possible to overcome this constraint by making provision for the detection by the detectors 10 to take place only after the autofluorescence signal emitted by the sample 2 (even presenting a spectrum superposed on the spectrum of the luminescence signal) has dissipated in the enclosure 5.

FIG. 6 shows a second embodiment of the invention which is applicable both when a continuous light source is used and when a pulsed light source is used, such sources being as described above with reference to FIGS. 1 to 5c. In the second embodiment, a separator plate 12 is not necessarily used, and the combined light signal is separated fully by the filter 13. However, due to the offset in position of the information detected by the first and by the second detectors 10 and 11, provision is made for a reconstruction unit of the processor unit 15 to be pre-calibrated so as to transpose the images obtained by the two detectors into a common reference frame, which can be the reference frame associated with either of the detectors, or some other reference frame.

Detection is performed by the first and second detectors 10, 11 during a period of observation throughout which the enclosure, containing the sample 2, is kept closed, it being possible for said sample to move inside the enclosure. During said period of observation, a plurality of time frames are defined during which detection takes place. The signal detected by each detector during each time frame can be converted directly into a luminescence image (for the first detector) or into a cinematographic image (for the second detector) for each time frame. As an alternative, and as described with reference to FIG. 4, the above-mentioned images are obtained for each time frame by computation on the basis of detection performed during an observation sub-period made up of a plurality of time frames.

Each detector can have a plurality of pixels organized as rows and columns in a plane of the detector, each pixel being identifiable by its (x,y) co-ordinates in said plane relative to an origin. Each pixel of co-ordinates (x,y) detects a signal coming from a region of co-ordinates (Du, Dv, Dw) of the enclosure corresponding, for example, to a cone whose base is formed by the (x,y) pixel.

The apparatus shown in FIG. 6 makes it possible to implement the method described for the first embodiment, in its first variant, with reference to FIG. 2. In the second embodiment of the apparatus, if it is desired to implement the method described with reference to FIGS. 5a to 5c, it is optionally possible to omit the filter 13.

In the above, in order to obtain the cinematographic image, a light signal is used that is emitted by the sample 2, such as, in particular, the light reflected by the sample from the light emitted by the light source 8.

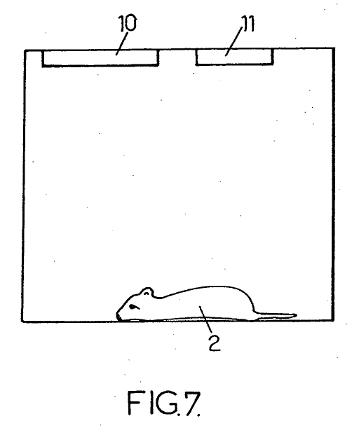

However, in the method described herein, the signal making it possible to obtain information on the position of the sample in the enclosure is not necessarily an optical signal. In the third embodiment of the invention, shown in FIG. 7, any type of detector making it possible to obtain information on the position of the sample 2 in the enclosure is used for the first detector 10. Such a detector can, for example, be constituted by a thermal detector adapted to detect heat given off from the mammal 2. Therefore, in such an embodiment, it is no longer necessary to illuminate the sample 2 by means of a light source 8. In addition, the luminescence signal detected by the second detector 11, and the heat signal detected by the first detector 10 are so different that the separation of the signals takes place naturally by using detectors of different types. The thermal detector is not disturbed by the luminescence signal emitted by the sample and the detector of the luminescence signal is not disturbed by the heat signal emitted by the sample.

1. Luminescence imaging apparatus comprising:
a light-tight enclosure containing a stage adapted to receive a sample that is to be imaged and that emits a first light signal carrying luminescence information about the sample;
a light source adapted to generate incident illumination towards the stage, the interaction between said incident illumination and the sample forming a second light signal;
detection apparatus adapted firstly to detect light signals presenting a luminescence spectrum and to store a first image on the basis of the light signals presenting a luminescence spectrum, and secondly to detect light signals corresponding to the reflection on the sample of the incident illumination coming from said light source and to store a second image on the basis of the light signals corresponding to the reflection on the sample of the incident illumination coming from said light source; and
an electronic control unit adapted to define a plurality of time frames, each time frame lasting for a length of time corresponding to acquiring and storing the second image;
said electronic control unit also being adapted to cause the light source to generate incident illumination during each time frame; and
a combined light signal corresponding to a combination of the first and second light signals reaching the detection apparatus during each time frame;
said luminescence imaging apparatus being characterized in that it further comprises separator means adapted so that, during each time frame, the detection apparatus acquires both first data relating to the luminescence information, and also second data relating to the second light signal.

2. Imaging apparatus according to claim 1, in which the detection apparatus has a plurality of pixels, each of which is adapted to detect light signals coming from a respective given region of the enclosure.

3. Imaging apparatus according to claim 1 or claim 2, in which the detection apparatus is adapted to store the first image on the basis of a first sampling of time frames and to store the second image on the basis of a second sampling of time frames, the first sampling having a frequency that is different from the frequency of the second sampling.

4. Imaging apparatus according to claim 3, in which the frequency of the first sampling is lower than the frequency of the second sampling.

5. Imaging apparatus according to any preceding claim, in which the second light signal comprises a light signal relating to the reflection on the sample of the incident illumination coming from said light source, and a light signal of autofluorescence of the sample subjected to said incident illumination, and in which the imaging apparatus is adapted to separate the light signals presenting a luminescence spectrum from the light signal of autofluorescence and from the light signal relating to the reflection.

6. Imaging apparatus according to any preceding claim, in which the detection apparatus comprises:
a first detector adapted to detect light signals carrying luminescence information; and
a second detector adapted to detect light signals corresponding to the reflection on the sample of the incident light coming from said light source.

7. Imaging apparatus according to claim 6, in which the separator means comprise a filter disposed at the inlet of the first detector, said filter being adapted to ensure that the light signals corresponding to the reflection on the sample of the incident light coming from said light source are not being acquired by the first detector.

8. Imaging apparatus according to claim 7, in which the separator means further comprise a separator plate adapted to transmit the first light signal to the first detector, and to transmit the second light signal to the second detector.

9. Imaging apparatus according to claim 7, in which the first and second detectors are offset angularly relative to each other, and each of said detectors receives the combined light signal directly, the imaging apparatus further comprising a reconstruction unit adapted to associate the first data and the second data with a reference frame associated with the enclosure.

10. Imaging apparatus according to any one of claims 7 to 9, in which the light source emits continuously, and in which the combined light signal is a spectral combination of the first and second light signals.

11. Imaging apparatus according to any one of claims 7 to 10, in which the first light signal presents a spectrum distributed between a shortest wavelength and a longest wavelength, and in which the light source emits an incident illumination distributed substantially beyond said longest wavelength.

12. Imaging apparatus according to claim 6, in which the separator means comprise a sequencer adapted so that the control unit causes the light source to generate said incident illumination in pulsed manner, each time frame presenting an ON time, during which the light source emits, and an OFF time, during which the light source does not emit;
the combined light signal being a temporal combination of the first and second light signals;
the sequencer being adapted to cause the first detector to be in a detection state during the OFF time, and to be in a non-detection state during the ON time.

13. Imaging apparatus according to any preceding claim, further comprising a processor unit adapted to transpose the luminescence information to a reference frame associated with the sample.

14. A method of performing luminescence imaging, said method comprising the following steps:
with a light-tight enclosure containing a stage receiving a sample that is to be imaged and that emits a first light signal carrying luminescence information about the sample;
(a) having an electronic control unit define a plurality of time frames, and having said control unit cause the light source to generate incident illumination towards the stage during each time frame, the interaction between said incident illumination and the sample forming a second light signal;
a combined light signal corresponding to a combination of the first and second light signals reaching detection apparatus during each time frame;
(b) separating the combined light signal so that, during each time frame, the detection apparatus, which is adapted firstly to detect light signals presenting a luminescence spectrum and to store a first image on the basis of the light signals presenting a luminescence spectrum, and secondly to detect light signals corresponding to the reflection on the sample of the incident illumination coming from said light source and to store a second image on the basis of the light signals corresponding to the reflection on the sample of the incident illumination coming from said light source, acquires both first data relating to the luminescence information, and also second data relating to the second light signal;
each time frame lasting for a length of time corresponding to acquiring and storing a second image.

15. An imaging method according to claim 14, in which, during step (a), each time frame is subdivided into an ON time during which the light source emits, and an OFF time during which the light source does not emit;
the combined light signal being a temporal combination of the first and second light signals;
and, during step (b), a first detector adapted to detect light signals presenting a luminescence spectrum is caused to be in a detection state during the OFF time, and to be in a non-detection state during the ON time, and a second detector adapted to detect light signals corresponding to the reflection on the sample of the incident illumination is caused to be in a detection state at least during the ON time.

16. An imaging method according to claim 14, in which, during step (b), a first detector detects the first light signal carrying the luminescence information, and a second detector detects the light signal corresponding to the reflection on the sample of the incident light coming from said light source, while ensuring that the light signals corresponding to the reflection on the sample of the incident light coming from said light source do not reach the first detector.

17. An imaging method according to claim 14, in which the detection apparatus has a plurality of pixels, and in which, during each time frame, the first data and the second data is associated with co-ordinates of a region of the enclosure.

18. An imaging method according to claim 17, in which, for each time frame, the first data is transposed to a reference frame associated with the sample.

19. An imaging method according to claim 14, in which, prior to step (a), a chemical reaction is triggered inside the sample, said chemical reaction generating the first light signal, and in which, after step (b) information relating to the chemical reaction is extracted from the first data and the second data.

20. An imaging method according to claim 17, which, prior to step (a), further consists in:
illuminating at least one molecule adapted to emit a phosphorescence signal due to it being illuminated; and
inserting the illuminated molecule into the sample;
the first light signal corresponding to phosphorescence light emitted by the molecule from inside the sample.

21. A method of performing luminescence imaging, said method comprising the following steps:
(c) placing a sample to be imaged on a stage in a light-tight enclosure, said sample having an outside surface defining an inside, the inside of said sample emitting a light signal into the enclosure through the outside surface, the sample also emitting a second signal of a type different from the type of the light signal;
(d) for a period of observation throughout which the sample is contained in the enclosure, detecting said light signal and said second signal;
said method further comprising the following steps:
(e) on the basis of the detection of the light signal, and for at least first and second successive time frames of the period of observation, forming a luminescence image of the sample representing the light signal emitted from inside the sample; and
(f) on the basis of the detection of the second signal, and for at least said first and second time frames, forming a cinematographic image of the sample that represents the position of the sample in the enclosure.

22. A luminescence imaging method according to claim 21, in which, for each time frame, a luminescence image of the sample is formed directly by detecting the light signal; and
in which, for each time frame, a cinematographic image of the sample is formed directly by detecting the second signal.

23. A luminescence imaging method according to claim 21, in which, for each time frame, a cinematographic image of the sample is formed directly by detecting the second signal;
in which, during a sub-period of detection, included in the detection period, and including at least the first and second time frames, an accumulated light signal is detected; and
in which, said luminescence image is formed for each time frame by processing said light signal on the basis of said corresponding cinematographic images.

24. A luminescence imaging method according to claim 21, further consisting, for at least one time frame, and preferably for each time frame, in:
(g) displaying on a screen a superimposed image corresponding to the superposition of the cinematographic image and of the luminescence image for said time frame.

25. A luminescence imaging method according to claim 21, further consisting, for at least one time frame, and preferably for each time frame, in:
(h) identifying a region of the luminescence image; and
(i) by means of at least one cinematographic image, associating said region with a zone of the sample.

26. A luminescence imaging method according to claim 25, further consisting, for said at least one time frame, and preferably for each time frame, in (j) obtaining a parameter relating to a chemical entity located in said zone on the basis of the luminescence image.

27. A luminescence imaging method according to claim 21, in which, during step (d), the light signal is detected at a first angle of incidence, and the second signal is detected at a second angle of incidence that is distinct from the first angle of incidence, and in which, for each time frame during steps (e) and (f), the luminescence image and the second image are formed in the same reference frame.

28. A luminescence imaging method according to claim 21, in which each image comprises a plurality of pixels (x,y) each of which corresponds to a zone (Du, Dv, Dw) of the enclosure.

29. A luminescence imaging method according to claim 21, in which the luminescence signal presents a spectrum, and in which the second signal is a light signal presenting a spectrum remote from the spectrum of the luminescence signal.

30. A luminescence imaging method according to claim 21, in which each time frame presents an ON time during which the sample is illuminated and during which the second signal is detected, and an OFF time during which the sample is not illuminated and during which the first signal is detected, and in which the second signal is a light signal corresponding to the reflection on the sample of the illumination.

31. A luminescence imaging method according to claim 21, in which the second signal is a thermal signal.

32. An imaging method according to claim 21, in which each time frame lasts for a length of time corresponding to the detection time of the second signal.
